(19) United States
(12) Patent Application Publication
(10) Pub. No.: US 2012/0143263 A1
(43) Pub. Date: Jun. 7, 2012

(54) SCREW

(75) Inventor: M. Ali Darendeliler, Kensington (AU)

(73) Assignee: UNIVERSITY OF SYDNEY, SYDNEY (AU)

(21) Appl. No.: 13/376,285

(22) PCT Filed: Jun. 4, 2010
(86) PCT No.: PCT/AU10/00695

§ 371 Date: Feb. 16, 2012

(30) Foreign Application Priority Data
Jun. 5, 2009 (AU) 2009902585

(51) Int. Cl. A61B 17/86

(52) U.S. Cl. 606/304; 606/309; 606/305

(57) ABSTRACT

An orthodontic mini-screw comprises a screw head arranged to be coupled to an ancillary member. A threaded body couples to the screw head and has a first portion arranged to locate in a cortical bone layer of a patient's jaw bone. A second portion of the threaded body is arranged to extend a predefined distance beyond the cortical bone layer and into an adjacent cancellous bone layer, such that a channel extends through the threaded body and out of at least one void located in the first portion. In use the channel is arranged to deliver a settable bio-compatible material into the cancellous bone layer to assist with anchorage of the mini-screw.

### TECHNICAL FIELD OF THE INVENTION

The present invention relates generally to a screw and more particularly, but by no means exclusively, to orthodontic mini-screws and anchorage systems for the same.

### BACKGROUND OF THE INVENTION

In the orthodontic field it is often necessary to utilise anchorage points to achieve sufficient forces for affecting tooth movement or for correcting orthodontic conditions such as overbite, jaw misalignment, etc.

In prosthodontics, permanent implants have been widely used since the late 1980s and market surveys project the accumulating number of dental implants to reach in excess of 200 million by 2016. However a problem with such implants is that clinicians have to wait at least 3 months after placement of the implant to allow osteointegration (bone growing around the implant) before they can be for anchorage (e.g. to place crowns on them, etc.). During the 3 month waiting time, replacement/provisional crowns are required, which causes extra cost and discomfort to the patients.

Two basic types of implant are currently used for orthodontic anchorage, namely integrated implants and non-integrated implants. Integrated anchorage implants are characterised by the implant fixture being in very close proximity to the enclosing bone, whereas non-integrated implants can be located in any bone adjacent to suitable mucosa, thereby increasing the range of possible locations for insertion. Further, non-integrated implants have a relatively less complex insertion procedure than integrated implants and allow for immediate loading.

One common type of non-integrated implant is the temporary anchorage device. Such devices include mini-screws, mini-pins, micro-screws and anchorage plates all of which can be fixed to suitable bone and comprise an end which extends out from the gum of a patient to be used for anchorage.

Mini-screws, as their name suggests, are miniature screws which can be temporarily fixed to the bone for the purpose of enhancing orthodontic anchorage. Mini-screws can be placed in a variety of locations in a patient's jaw but are usually located between or near the roots of the patient's teeth or in the patient's palate.

Due to their stability and wide range of possible insertion locations, mini-screws are particularly useful for closing gaps due to missing teeth, distalising or retracting teeth, intruding over-erupted teeth, reducing occlusal plane cants, as anchorage to orthopaedic and/or inter-maxillary forces, and other applications.

Mini-screws can be used as either direct or indirect anchorage points. Direct anchorage refers to applying an active force directly from the mini-screw to the “active unit” (e.g. tooth or teeth intended to be moved). Indirect anchorage on the other hand is used to describe situations where the mini-screw is used to stabilise a tooth or group of teeth to prevent side effects and allowing conventional mechanics to be used with no anchorage concern.

Mini-screws for orthodontic applications include a head arranged to couple to the active unit and a threaded screw body that is typically between 6 mm to 12 mm in length. While longer screws (i.e. 8 mm to 12 mm) are generally considered to exhibit better mechanical stability, the disadvantage is that there is a greater risk of invading adjacent anatomical structures, such as roots and nerve endings. For typical cortical bone applications a screw thread length of 6 mm is usually deemed appropriate. However, even with 6 mm screws, there is still a risk of damaging adjacent nerves etc if insufficient attention is paid to their placement.

### SUMMARY OF INVENTION

In accordance with a first embodiment of the present invention there is provided an orthodontic mini-screw comprising:
a screw head arranged to be coupled to an ancillary member; and
a threaded body coupled to the screw head and having a first portion arranged to locate in a cortical bone layer of a patient's jaw bone and a second portion arranged to extend a predefined distance beyond the cortical bone layer and into an adjacent cancellous bone layer, such that a channel extends through the threaded body and out of at least one void located in the first portion, in use the channel being arranged to deliver a settable bio-compatible material into the cancellous bone layer to assist with anchorage of the mini-screw.

In an embodiment the at least one void is located in a circumferential wall of the threaded body.

In an embodiment the at least one void is located between a thread disposed on the threaded body.

In an embodiment the mini-screw comprises a plurality of voids laterally displaced along the second portion of the threaded body.

In an embodiment a distal end of the threaded body comprises a solid cutting tip.

In an embodiment the second portion extends between 1 mm and 4 mm into the cancellous bone layer.

In an embodiment the second portion extends between 2 mm and 3 mm into the cancellous bone layer.

In an embodiment the threaded body has a total length of between 5 mm and 7 mm.

In an embodiment the at least one void is located at a bone engaging end of the threaded body.

In an embodiment at least one additional void is provided on a circumferential wall of the first portion. In this manner, the bio-compatible material can additionally be applied to the cortical bone layer.

In an embodiment the channel extends the length of the threaded body such that a needle of a syringe containing the bio-compatible material can be inserted into the cavity for injecting the bio-compatible material into the cancellous (and optionally cortical) bone layer.

In an embodiment the channel has a retentive inner wall that allows bio-compatible material, once set, to be retained thereto.

In an embodiment the bio-compatible material comprises a resorbable material.

In accordance with a second aspect of the present invention there is provided an orthodontic mini-screw comprising:
a screw head arranged to be coupled to an ancillary coupling member; and
a threaded body, the length of which substantially corresponds to a thickness of a layer of cortical bone in which the mini-screw is to be located.

In an embodiment the length of the threaded body ranges from between 1 mm and 5 mm, for a typical cortical bone thickness. In another embodiment, the length is between 2 mm and 4 mm. In an embodiment the length is approximately 3 mm. It will be understood, however, that in some cases the cortical bone may be particularly thick and a longer screw thread may be required.

In an embodiment the mini-screw comprises an anchorage device which, in use, engages a wall of the cortical bone to prevent the mini-screw from becoming unintentionally loosened.

In an embodiment the anchorage device comprises at least one anchorage wire which is arranged to extend out of an opening in a distal end of the thread body and into a second non-cortical bone layer before engaging an internal cortical bone wall.

In an embodiment the opening extends into a peripheral wall of the threaded body to allow the anchorage wire(s) to immediately extend out of the threaded body into the second layer. Such a configuration allows for minimal intrusion by the wires into the secondary bone layer, even where part the distal end of the mini-screw thread slightly extends into secondary layer.

In an embodiment the second non-cortical bone layer is composed of cancellous bone.

In an embodiment the at least one anchorage wire is formed of super-elastic shape memory material which allows the wire, having passed out of the distal end, to form a shape which is suitable for engaging the internal wall with minimal disruption or intrusion to the cancellous bone layer.

In an embodiment the shape is a generally parabolic shape. Alternatively, when two wires are utilised, their respect ends extend into the second layer so as to substantially form an oval shape within the second layer.

In an embodiment the elasticity of the super-elastic wire is selected so as to allow the wire to take the desired shape with minimal diversion during emplacement.

In an embodiment the super-elastic shape memory base material is NiTi.

In an embodiment the anchorage device comprises at least one projection which, in use, extends out of a circumferential wall of the threaded body to thereby engage the internal cortical bone wall.

In an embodiment the screw head includes a cavity arranged to retain a non-engaging end of the anchorage wire(s). In an embodiment a head portion of the cavity is threaded such that the non-engaging end of each anchorage wire is coupled to a retaining screw that is operable to be screwed in and out of the threaded cavity to thereby engage and disengage, respectively, the anchorage wires.

It will be understood by persons skilled in the art that the anchorage wires can be inserted during or after the placement of the mini-screw and equally can be pulled back before or during the removal of the screw.

In an embodiment the mini-screw further comprises a collar located between the threaded body and screw head, such that the threaded cavity is at least partly located within the collar.

In an embodiment the threaded body includes a cavity which is open at a bone engaging end, the cavity being arranged to receive and deliver a settable bio-compatible material into a cancellous bone layer behind the cortical bone, to thereby assist with anchorage of the mini-screw.

In an embodiment the cavity extends the length of the threaded body such that a needle of a syringe containing the bio-compatible material can be inserted into the cavity for injecting the bio-compatible material into the cancellous bone layer.

In an alternative embodiment, the bio-compatible material may be stored in the in the cavity until such times as it is required to be injected into the cancellous bone layer. For example, a plunger type configuration may be provided within the screw such that once the plunger is actuated a seal located at the distal end of the screw thread (which is provided to prevent the bio-compatible material from setting when not in use) may break to allow the bio-compatible material to be forced into the cancellous layer.

In an embodiment the cavity has a retentive inner wall that allows bio-compatible material, once set, to be retained thereto.

In an embodiment the threaded body includes a cavity which is open at a bone engaging end and arranged to receive a deformable bladder filled with a bio-compatible fluid, such as saline or the like. In use at least part of the bladder arranged to extend into a cancellous bone layer behind the cortical bone layer, to thereby assist with anchorage of the mini-screw.

In an embodiment the mini-screw further comprises a plunger arranged to force the bladder into the cortical bone layer. In an embodiment the plunger is coupled to the head of the mini-screw.

In an embodiment the plunger comprises a head that is received by a threaded head portion of the mini-screw and a stem coupled to the bladder and arranged to extend into the cavity, such that rotation of the head causes the bladder to either move into or out of the cancellous bone layer. The aforementioned plunger configuration may also be used for the pre-stored bio-compatible material embodiment mentioned above.

In accordance with a third aspect of the present invention there is provided an orthodontic treatment method requiring cortical bone anchorage comprises: screwing a mini-screw in accordance with the first aspect into an insertion location using the screw head; and passing the settable bio-compatible material through the channel and into the cancellous bone layer.

In accordance with a fourth aspect the present invention provides an orthodontic treatment method requiring cortical bone anchorage, the method comprising providing a mini-screw comprising:
a screw head arranged to be coupled to an ancillary coupling member for affecting the treatment; and
a threaded body, the length of which substantially corresponds to a thickness of the cortical bone at an insertion location; and
screwing the mini-screw into the insertion location.

In an embodiment the method comprises the further step of actuating an anchorage device received by the threaded body such that it engages an internal wall of the cortical bone to prevent the mini-screw from becoming loosened.

In an embodiment the anchorage device comprises at least one anchorage wire which is arranged to extend out of an opening in a distal end of the threaded body and into a second non-cortical bone layer before engaging the internal cortical bone wall.

In an embodiment the opening extends into a wall of the threaded body to allow the anchorage wire(s) to immediately extend out of the threaded body into the second layer.

In an embodiment the second non-cortical bone layer is composed of cancellous bone.

In an embodiment the at least one anchorage wire is formed of super-elastic shape memory material which allows the wire, having passed out of the distal end, to form a shape which is suitable for engaging the internal wall with minimal disruption to the cancellous bone layer.

In an embodiment the shape is a generally parabolic shape. Alternatively, when two wires are utilised that make extend into the second layer so as to substantially form an oval shape within the second layer.

In an embodiment the screw head includes a cavity arranged to retain an outer end of the anchorage wire(s). In an embodiment the cavity is threaded such that a non-engaging end of each wire is coupled to a retaining screw such that the step of actuating the anchorage device comprises screwing the retaining screw into the threaded cavity.

In an embodiment the threaded body includes a cavity which is open at a bone engaging end, the cavity being arranged to receive and deliver a settable bio-compatible material into a cancellous bone layer behind the cortical bone, to thereby assist with anchorage of the mini-screw. According to such an embodiment the treatment method involves injecting the bio-compatible material into the cancellous bone layer.

In an embodiment the cavity extends the length of the threaded body such that a needle of a syringe containing the bio-compatible material can be inserted into the cavity for injecting the bio-compatible material into the cancellous bone layer.

In an alternative embodiment, the bio-compatible material may be stored in the in the cavity until such times as it is required to be injected into the cancellous bone layer. For example, a plunger type configuration may be provided within the screw such that the method involves actuating the plunger which in turn causes a seal located at the distal end of the screw thread (which is provided to prevent the bio-compatible material from setting when not in use) to break thereby allowing the bio-compatible material to be forced into the cancellous layer.

In an embodiment the cavity has a retentive inner wall that allows bio-compatible material, once set, to be retained thereto.

In an embodiment the threaded body includes a cavity which is open at a bone engaging end and arranged to receive a deformable bladder filled with a bio-compatible fluid, such as saline or the like. According to such an embodiment, the treatment method involves applying pressure to the bladder causing at least part of the bladder to extend into a cancellous bone layer behind the cortical bone layer, to thereby assist with anchorage of the mini-screw.

In accordance with a fifth aspect of the present invention there is provided a screw comprising:
a screw head;
a threaded body for screwing into an anchorage layer; and
an anchorage device comprising at least one anchorage wire arranged, in use, to extend out of an opening in the threaded body and into a space behind the anchorage layer, such that at least part of the anchorage wire engages an internal wall of the anchorage layer to thereby assist with anchorage of the screw.

In accordance with a sixth aspect there is provided a screw comprising:
a screw head; and
a threaded body for screwing into an anchorage layer, wherein the threaded body includes a cavity, in use, arranged to receive and deliver a settable substance into a space behind the anchorage layer, to thereby assist with anchorage of the screw.

In an embodiment the substance comprises at least one of silicon, expandable foam and industrial cement.

In an embodiment the cavity extends the length of the threaded body such that a needle of a syringe containing the settable substance can be inserted into the cavity for injecting the substance into the space. In an embodiment the space has a material density.

In an embodiment the cavity has a retentive inner wall that allows the substance, once set, to be retained thereto.

In accordance with a seventh aspect the present invention provides a screw comprising:
a screw head; and
a threaded body for screwing into an anchorage layer, wherein the threaded body includes a cavity arranged to receive a deformable bladder filled with a fluid, in use, at least part of the bladder arranged to extend into a space behind the anchorage layer, to thereby anchor the screw.

In an embodiment the screw further comprises a plunger arranged to force the bladder into the space.

In an embodiment the plunger is coupled to the head of the mini-screw.

In an embodiment the plunger comprises a head that is received by a threaded head portion of the mini-screw and a stem coupled to the bladder and arranged to extend into the cavity, such that rotation of the head causes the bladder to either extend into or out of the space.

In accordance with a eighth aspect of the present invention there is provided a mini-screw arranged to be located in a cortical bone layer, the mini-screw comprising:
a screw head arranged to be coupled to an ancillary coupling member; and
a threaded body providing an anchorage device which, in use, engages an internal wall of the cortical bone to prevent the mini-screw from becoming loosened.

In an embodiment a channel extends at least partially though the threaded body and out at least one void located in a circumferential wall of the threaded body for delivering a substance to the cortical bone. In an embodiment the substance is a bio-comptatible settable cement.

In an embodiment the anchorage device comprises at least one anchorage wire which is arranged to extend out of an opening in a distal end of the threaded body and into a second non-cortical bone layer, before retuning to engage the first layer's internal wall.

In an embodiment the opening extends into a wall of the threaded body to allow the anchorage wire(s) to extend out of the threaded body immediately into the second layer.

In accordance with a ninth aspect of the present invention there is provided an orthodontic treatment method requiring cortical bone anchorage, the method comprising
determining a thickness of the cortical bone;
selecting a mini-screw comprising:
   a screw head arranged to be coupled to an ancillary coupling member for affecting the treatment; and
   a threaded body having a length which substantially corresponds to the determined thickness; and
screwing the mini-screw into the insertion location.

In accordance with a tenth aspect of the present invention there is provided a medical screw for anchoring in the body, the medical screw comprising:
a threaded body providing an anchorage device which, in use, engages an internal wall of a first bone layer to prevent the mini-screw from becoming loosened.

In accordance with a eleventh aspect the present invention provides a prosthodontic implant comprising:
a head arranged to be coupled to a prosthodontic appliance; and
a body for locating into an anchorage layer, wherein the body includes a channel extending at least partially through the body and out of at least one void located in a circumferential wall of the body, in use the channel arranged to deliver a substance to the anchorage layer.

In an embodiment the implant is a screw having a threaded body. In an embodiment the substance is a bio-compatible material that assists in anchorage of the prosthodontic implant. The bio-compatible material may be settable cement. In an embodiment, the settable cement is resorbable for temporary anchorage of the prosthodontic implant. Alternatively, for more permanent applications, the settable cement may be non-resorbable.

In an embodiment the prosthodontic appliance may be a tooth, teeth, denture, or other suitable prosthodontic appliance.

### BRIEF DESCRIPTION OF THE FIGURES

Features and advantages of the present invention will become apparent from the following description of embodiments thereof, by way of example only, with reference to the accompanying drawings, in which:

FIG. 1 is a stress distribution diagram illustrating stresses applied by a conventional mini-screw in both cortical and cancellous bone around an implant location;

FIG. 2a is a schematic side view of a mini-screw anchored in a first layer of cortical bone, in accordance with an embodiment of the present invention;

FIG. 2b shows the mini-screw of FIG. 2a coupled to an ancillary configuration, in accordance with an embodiment of the present invention;

FIG. 2c is a stress distribution diagram for the mini-screw of FIG. 2a;

FIGS. 3a and 3b are sectional schematic views of a mini-screw incorporating an anchorage device in pre and post activation state, respectively;

FIG. 3c is a stress distribution diagram for the mini-screw shown in FIGS. 3a and 3b;

FIG. 4 is an end view of the FIG. 3 mini-screw head incorporating the anchorage device;

FIGS. 5a and 5b are sectional side views of an alternative mini-screw incorporating an early actuator slot, showing the anchorage wires in both a non-actuated and actuated state respectively;

FIGS. 6a and 6b are front and side sectional views respectively of an embodiment showing a screw-threaded engaging mechanism for actuating the anchorage wires;

FIG. 7 shows an oval-shaped anchorage wire configuration, pre and post activation of the anchorage device;

FIG. 8 is a schematic of a mini-screw in accordance with a further embodiment of the present invention;

FIGS. 9a and 9b are sectional schematics of the FIG. 8 screw illustrating delivery of a bio-compatible material to the cancellous layer.

FIG. 10a is a front view of an alternative embodiment of the FIG. 8 mini-screw;

FIGS. 10b and 10c are sectional side views of the FIG. 10a embodiment showing injection of a bio-compatible fluid;

FIG. 10d is a sectional side view of the FIG. 10a embodiment showing removal of the mini-screw;

FIGS. 11a and 11b are sectional side views of a mini-screw incorporating a bladder, in accordance with an alternative embodiment; and

FIGS. 12 and 13 are schematics of a prosthodontic implant in accordance with an embodiment.

### DETAILED DESCRIPTION OF EMBODIMENTS

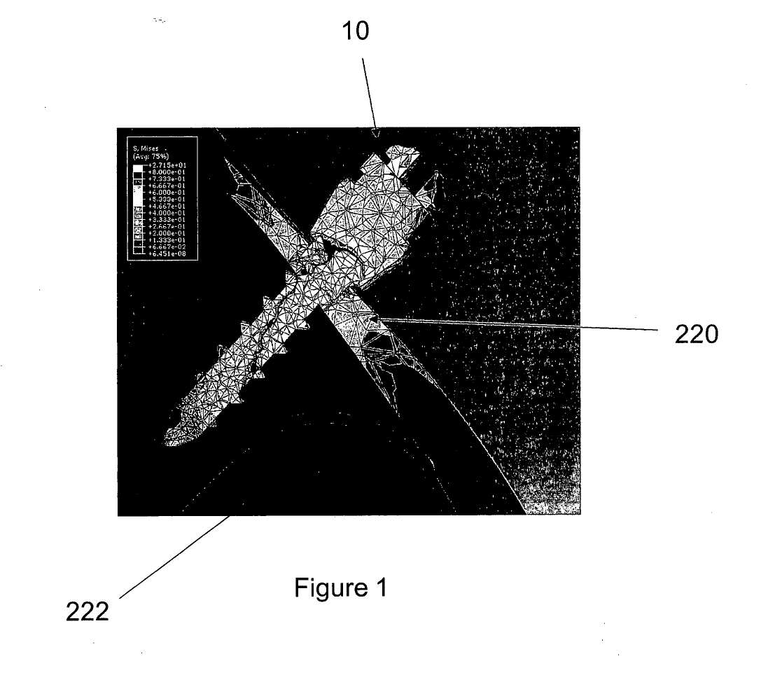

Embodiments described herein have been conceived based on the realisation by the present inventor that sufficient retention and stability of orthodontic mini-screws can be achieved by anchorage primarily in the first layer of a patient's cortical bone. With reference to the stress distribution diagram of FIG. 1, it can be observed that when a typical anchorage distribution force (i.e. a 300 gram horizontal force) is applied to the head of an in-situ mini-screw, roughly 99% of the stress is distributed in the cortical bone 220, with the remaining force distributed between the screw 10 and adjacent layer 222 of cancellous bone (indeed this graph effectively shows that the Von Mises stress in cortical bone is 100 to 500 times greater than the stress in the cancellous bone).

Embodiments draw on this realisation to provide a mini-screw that may comprise a significantly shorter screw thread length than conventional mini-screws and which thus presents less risk to root injury and nerve damage during emplacement. In addition the shorter thread length opens up the number of possible insertion sites. Embodiments also relate to anchorage systems for such shortened mini-screws. Furthermore, it has been realised that anchorage systems developed for use with the aforementioned orthodontic mini-screws may also be applied to larger screws (i.e. screws other than orthodontic mini-screws) for achieving appropriate anchorage, as will be described in more detail in subsequent paragraphs.

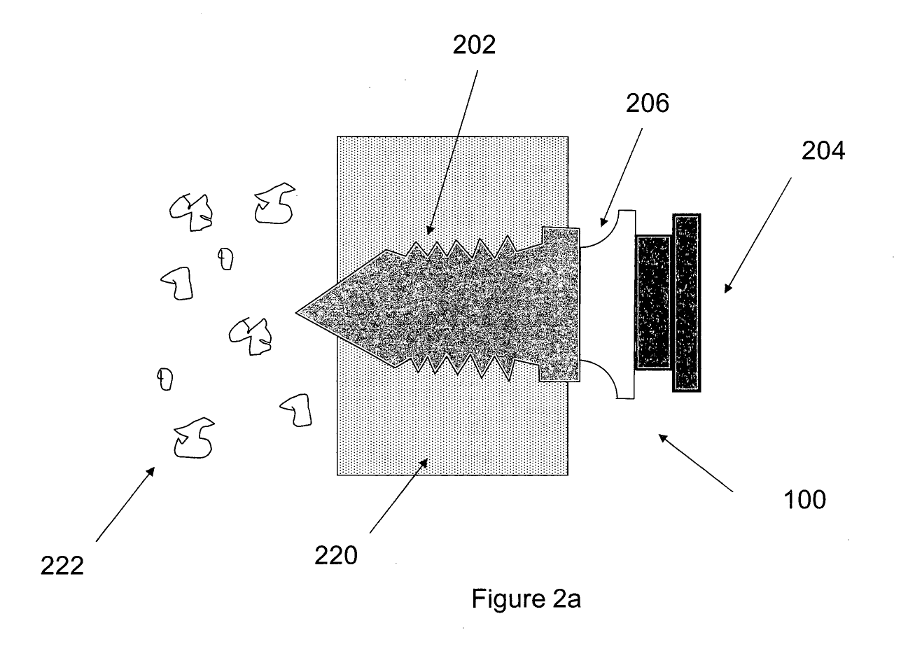

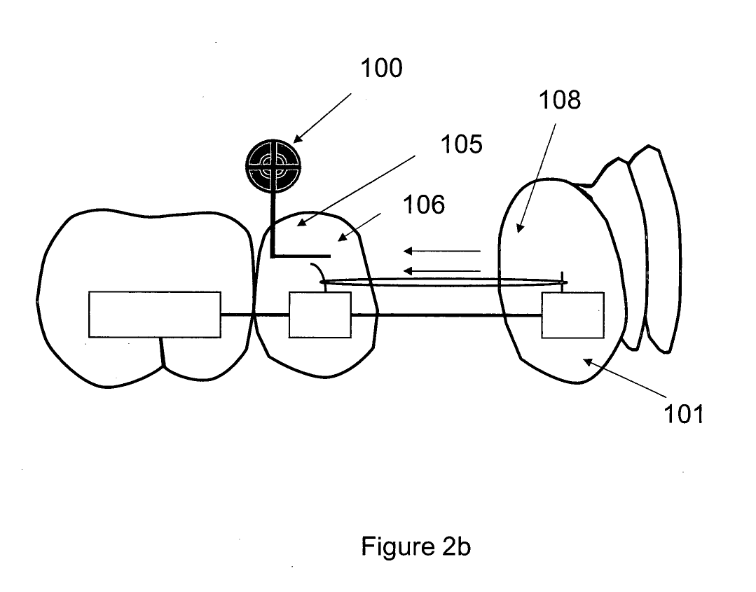

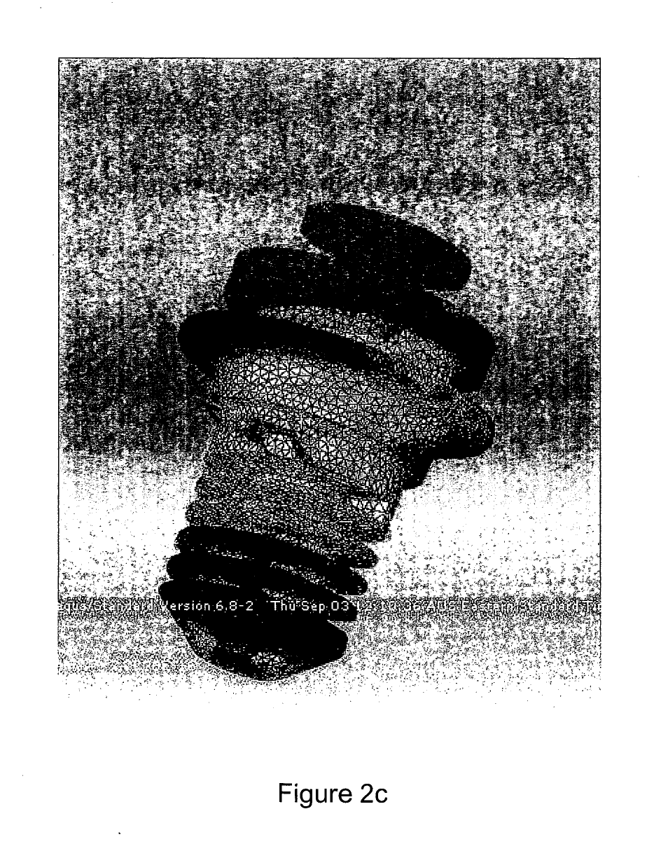

With reference to FIG. 2a there is shown a schematic of an orthodontic mini-screw 100, in accordance with a first embodiment of the present invention. The mini-screw 100 comprises a threaded shaft 202 for screwing into a first layer of cortical bone 220, or other suitable anchorage structure. It will be noted that the length of the threaded shaft 202 substantially corresponds to the width of the cortical bone layer 220 (as mentioned above, a shortened thread length ensures that damage to nerve and root endings located within the second cancellous bone layer 222 is avoided). In an embodiment, the cortical bone width may be determined, for example, by taking an x-ray of the patient's gum. Alternatively, the relative width may be estimated based on factors such as the bone region, age of the patient, etc. For orthodontic applications, the cortical bone thickness typically ranges between 0.8 mm and 5 mm (again depending on the area of jaw, age of the patient, etc.). Thus, mini-screws that have a length of between 0.8 mm and 6 mm may be suitable for such applications. An outwardly flanged head 204 couples to the threaded shaft 202 by way of a polished transmucosal collar 206. A distal end of the mini-screw 100 shown in FIG. 2a comprises a solid cutting tip for self drilling. It will be appreciated, however, that the distal end could have a flat profile for pre-drilling applications.

The mini-screw 100 is operable to be coupled to an orthodontic anchorage device for imparting a force on an orthodontic arrangement. One such example arrangement is shown in FIG. 2b where the mini-screw has been located in a patient's upper gum so as to provide absolute anchorage for facilitating movement of a patient's canine tooth 101. It will be appreciated by persons skilled in the art that the mini-screw 100 could be positioned in any suitable bone region that provides sufficient anchorage and is dependent only on the desired application. For example, the mini-screw could be positioned within a patient's gum, the palatal/lingual side of their jaw, or any other suitable region of bone. A stress distribution diagram for the FIG. 2b screw embodiment is shown in FIG. 2c.

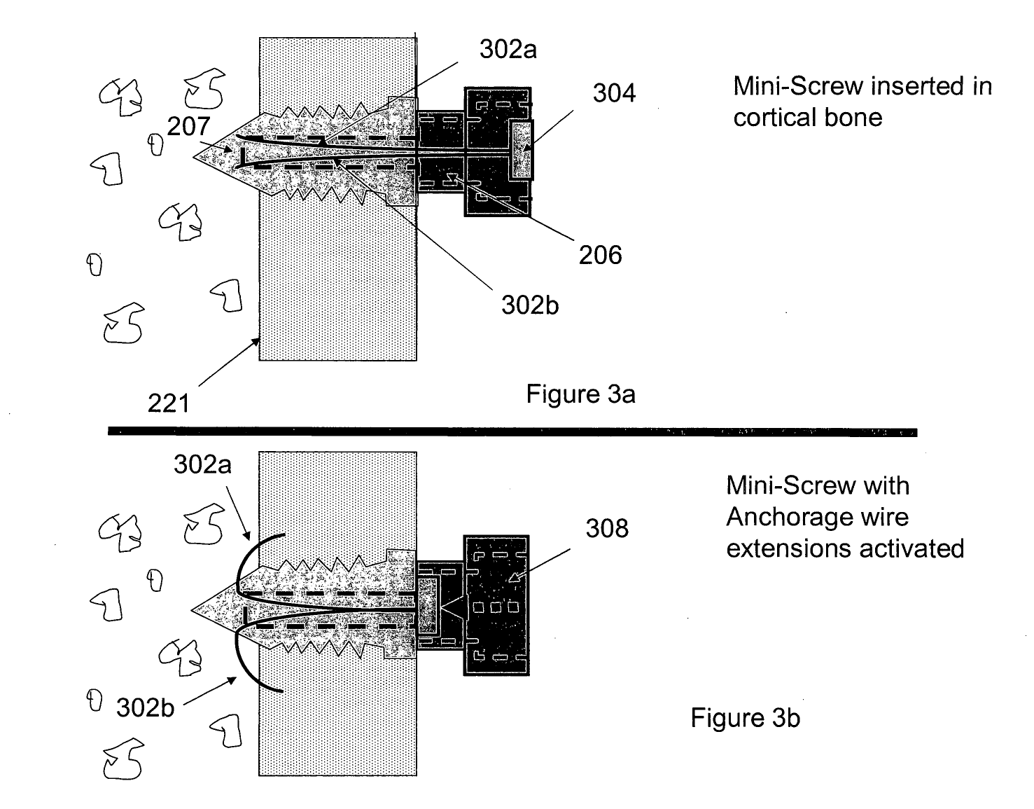

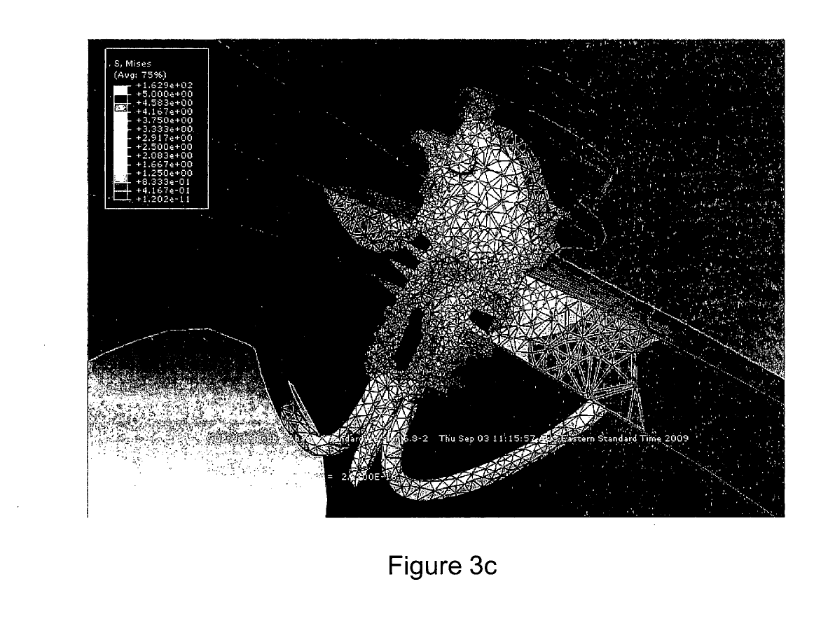

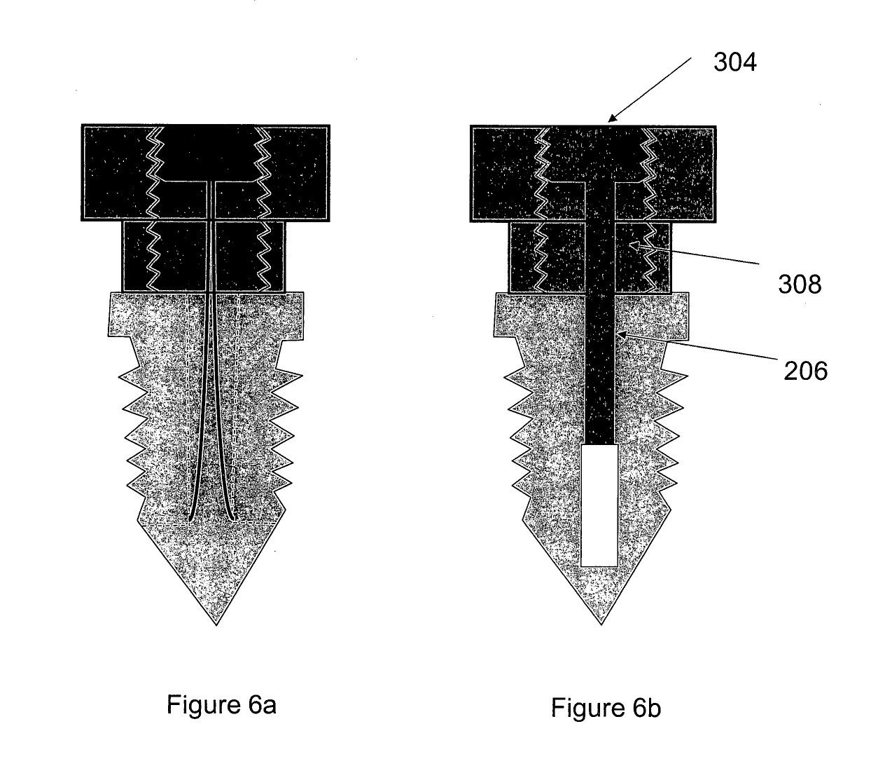

With reference to FIGS. 3a to 3c, an embodiment of the mini-screw 100 incorporates an anchorage device 302 which is operable to prevent the mini-screw 100 from become loosened, due to its short thread length. According to the FIG. 3 embodiment, the anchorage device 302 is received in a cavity 206 that extends through the body of the mini-screw 100 and comprises a plurality of anchorage wires 302a, 302b, 302c (while three anchorage wires are shown it will be understood that more or less wires may be used depending on the desired implementation) that have a pointed distal end which allows the wires to readily pierce and pass through the cancellous bone layer 222. At an opposite (non-engaging) end, the wires 302 are coupled to a threaded screw head 304, by way of a swivel. The part of the cavity in which the threaded screw head 304 is disposed (referred to herein as the head cavity and designated by reference numeral 308) comprises a corresponding thread for allowing the anchorage device to be actuated, as will be described in more detail in subsequent paragraphs. A schematic of the screw threaded engaging mechanism is best shown in FIG. 6.

Once the mini-screw 100 has been suitable located, the screw head 304 of the anchorage device 302 is turned causing the anchorage wires 302a, 302b, 302c to extend out of the distal opening 207 of the cavity 206 and into the cancellous bone 204. The swivel prevents the wires from twisting with the rotation of the screw head 304. A stress distribution diagram for this embodiment is shown in FIG. 3.

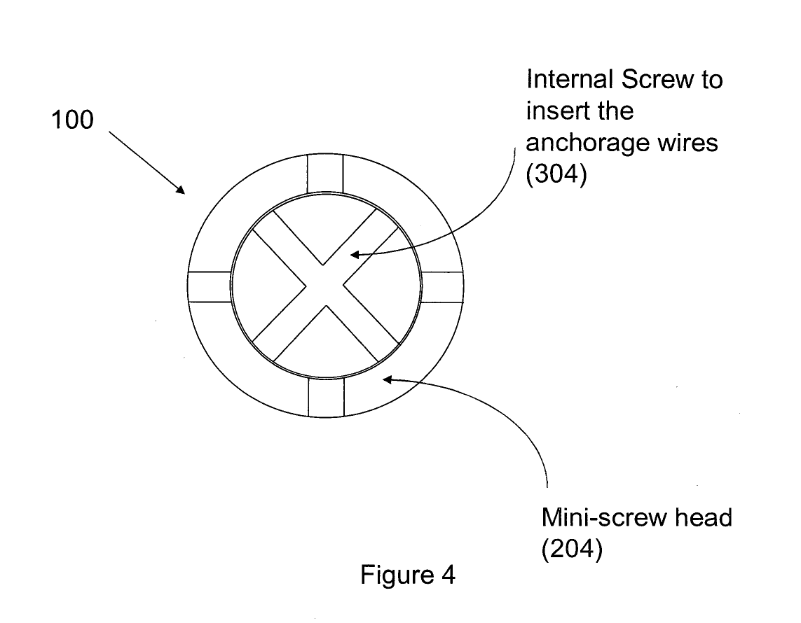

A plan view of the mini-screw head 204 incorporating the head 304 of the anchorage device 302 is best shown in FIG. 4. As will be readily appreciated, the tool (e.g. screwdriver) used to turn screw head 304 is required to have a slightly smaller head diameter than that which is used to turn the head of the mini-screw 204. It will be understood however that mechanisms other than a swivelled screw head 304 could equally be used for retaining the anchorage wires 302a, 302b. For example, the wires 302a, 302b may simply couple to a press-stud that can be pushed into the head cavity 308 for actuating the anchorage wires 302a, 302b. The press-stud may be retained in place by way of one or more deformable lugs located on an inner wall of the head cavity 308.

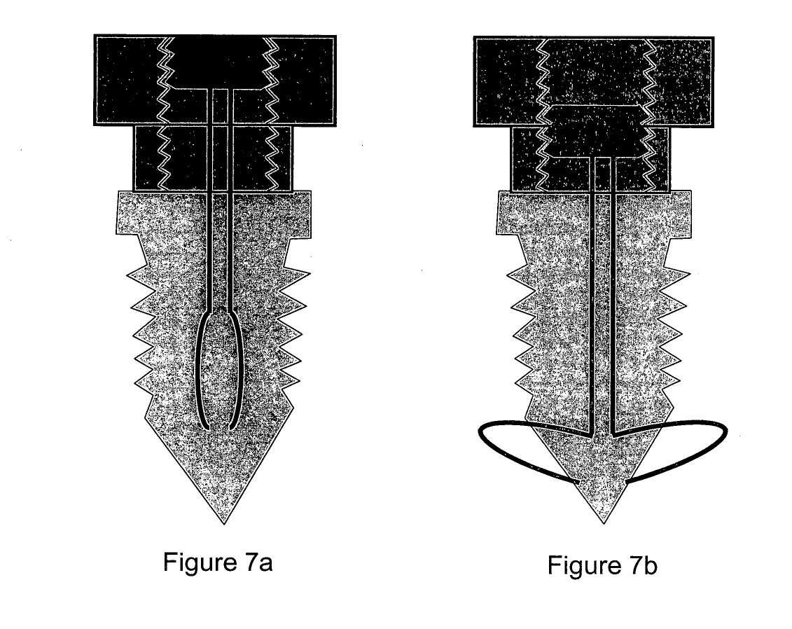

To minimise the risk of nerve and root damage, the wires 302a, 302b, 302c are formed such that immediately after passing outwardly through the distal end 207 of the mini-screw 100, they either tend away from one another and back toward an inner wall 221 of the cortical bone (see FIG. 3b), or alternatively toward one another so as to form the configuration illustrated in FIG. 7. In both cases, at least part of the anchorage wire 302 engages the inner wall 221 of the cortical bone 220 to thereby retain the mini-screw and prevent it from becoming loosened.

To achieve the various shapes described above, the anchorage wires 302a, 302b, 302c may be formed from a shape memory material that allows the wires to assume a desired shape after having been deformed (e.g. during retention in the void 206). Some suitable materials include copper-zinc-aluminium-nickel, copper-aluminium-nickel, and nickel-titanium (NiTi) alloys. The shape memory material may be selected such that it is activated by the heat present in the cancellous bone.

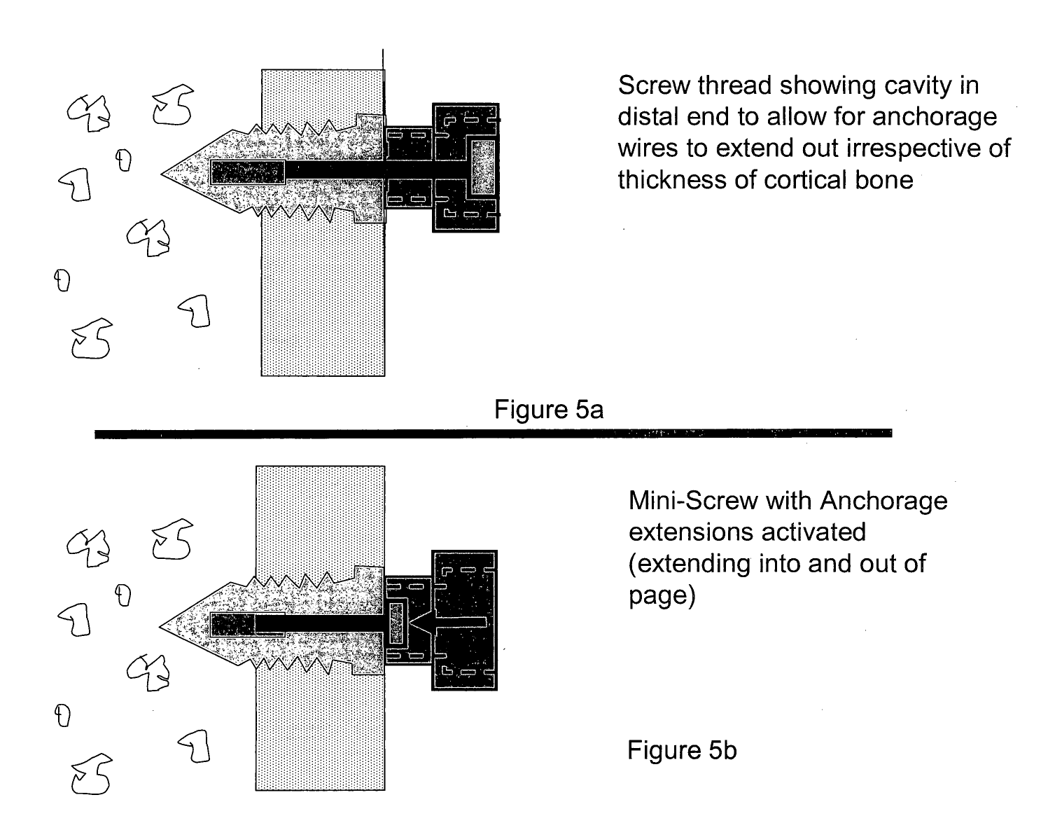

An alternative embodiment of the mini-screw is shown in FIG. 5. In this embodiment the opening 207 of the cavity 206 extends into a peripheral wall of the threaded body 202 to allow the anchorage wires 302 to immediately extend out of the threaded body into the cancellous bone layer, as soon as the distal end passes into the cancellous layer 222. As can be seen from FIG. 5b, the aforementioned configuration allows for minimal intrusion by the wires into the cancellous layer 222, even in situations where the distal end of the mini-screw thread slightly extends into cancellous layer.

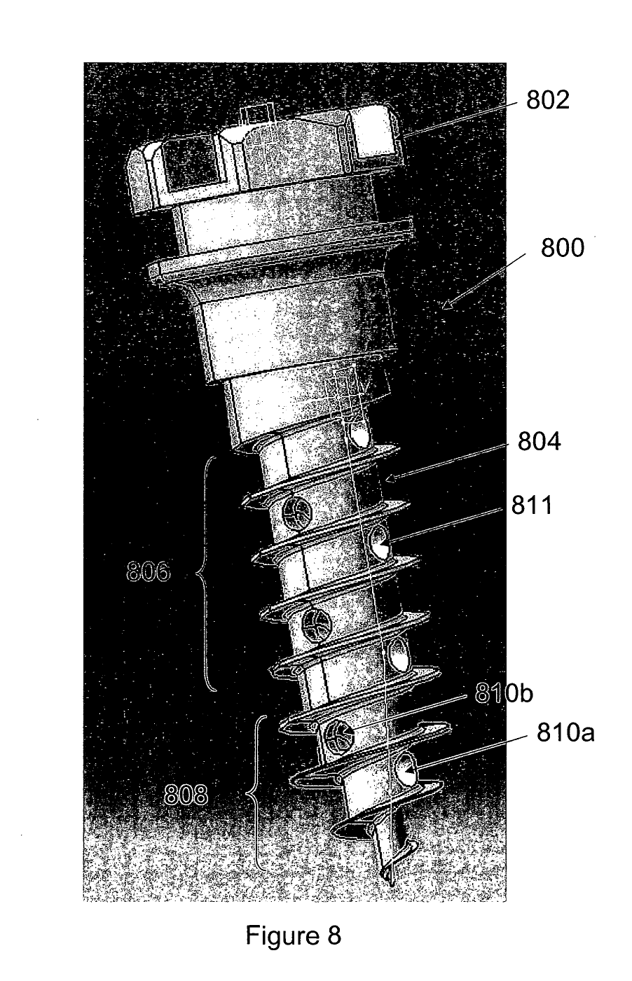

With reference to FIG. 8 there is shown a mini-screw 800 incorporating an anchorage mechanism in accordance with yet a further embodiment. In this embodiment the cavity 206 (hereafter channel) is not utilised to house anchorage wires, but instead to receive and deliver a settable bio-compatible material 801 into the cancellous bone layer 222, to thereby assist with anchorage of the mini-screw. The self setting bio-compatible material may take many forms, including but not limited to, tri calcium phosphate, bone graft material, hydroxyapatite cement, bone morphogenetic protein (BMP) containing grafting materials, or any other bio-friendly or bio-compatible material that sets in an appropriate time (e.g. 1 to 20 minutes, although some materials may take longer or shorter depending on the application and strength required).

As previously mentioned, embodiments take advantage of the realisation that the bulk of the force load for an orthodontic mini-screw is distributed through the dense cortical bone, and very little in the much less dense trabecular (cancellous) bone. By increasing the density of the cancellous bone using a bio-compatible reinforced material, two distinct advantageous may be achieved. First, the load may be distributed more evenly over the mini-screw thereby changing the ratio of trabecular/cortical contact to a system that resembles a very thick cortical plate. Secondly, increased lateral stability and pull out resistance can be achieved through having a bulbous mass engaging the internal surface of the cortical bone.

The bio-compatible materials 801 used for such an embodiment may have particular rheological properties such that the balance between compressive stress and the ability to flow (given the particularly narrow channel dimensions in the order of 1 mm) are suitable for such an orthodontic application. Osteoconductive and osteoinductive factors may be utilised to stimulate osteoblastic consolidation of the synthetic bone mass. It is also noted that such materials may be selected due to their resorbtion properties which allow the mini-screw to be readily removed after a desired treatment time. In other words, planned removal of the mini-screw can be effected by selecting a material having desired resorbtion properties. In this regard, a radiographic marker may be added to the material so that a technician can assess the extent of turnover and resorbtion. Further, in an embodiment, the material may be supplemented with a substance, such as bioactive glass, which may reduce inflammation and necrosis. In other words, in various embodiments, the material may comprise crystalline ceramic or calcium based material, binding polymers (of which the viscosity is critical), +/−bone factors, +/−anti inflammatory materials, +/−radiographic markers.

In more detail, the mini-screw 800 shown in FIG. 8 comprises a screw head 802 arranged to be coupled to an ancillary member. A threaded body 804 coupled to the screw head 802 has a first portion 806 arranged to locate in the cortical bone layer 220. A second portion 808 is arranged to extend a predefined distance (in the illustrated embodiment, approximately 2 mm so as to allow sufficient depth for distribution of the material 801) beyond the cortical bone 220 and into the adjacent cancellous bone 222. As previously mentioned, a channel 206 extends through the threaded body 804 and out at least one void 810 (in the illustrated embodiment two voids are shown although it will be understood that more or less voids may be provided depending on the desired implementation) located in a circumferential wall of the second portion 808. In the illustrated embodiment, the voids 810 are located between the threads disposed on the outer circumferential wall (although it will be understood that in an alternative embodiment they may locate through the threaded sections).

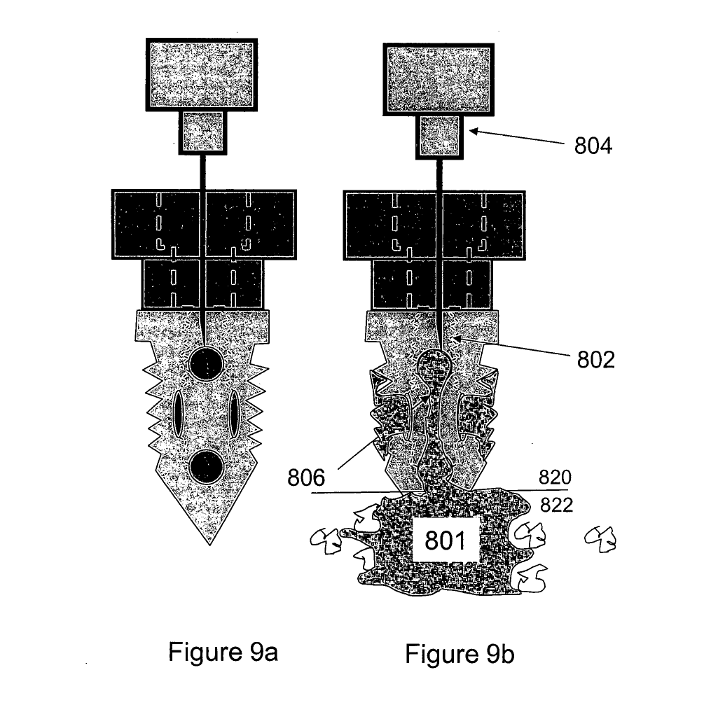

In use, cavity 206 is shaped so as to allow a needle 830 of syringe 832 containing the bio-compatible material 801 to be inserted into the cavity 206. The cavity 206 has a retentive inner wall 812 which allows the bio-compatible material 800, once set, to be retained thereto. Once set, the mechanical strength of the bio-compatible material is sufficient to avoid breakage under normal operating conditions (e.g. when experiencing typical rotational forces due to the ancillary acting on the tooth or teeth), but will break upon a sufficient torque being applied to the screw head during removal of the mini-screw. In the embodiment illustrated in FIG. 8, additional cavities 811 in communication with the channel 206 are provided in a circumferential wall of the first portion 806 to distribute the bio-compatible material 801 to the adjacent cortical bone layer 220 to further assist with anchorage of the screw. FIGS. 9a and 9b illustrate delivery of the bio-compatible material.

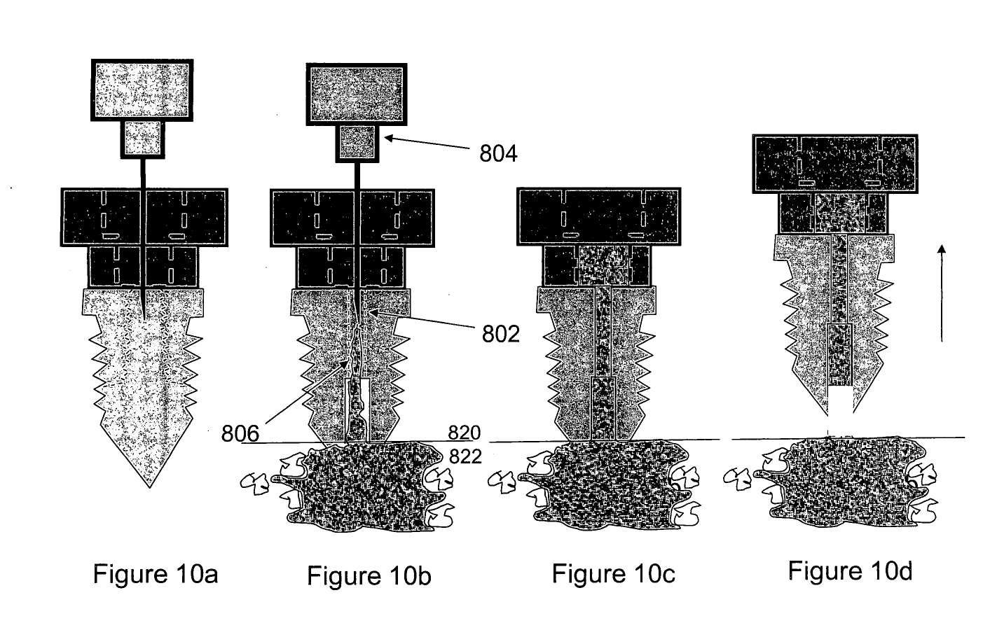

In an alternative embodiment to that shown in FIGS. 8 and 9, a single cavity may be provided at a distal end of the threaded body 804 for delivering the bio-compatible material 801 into the cancellous layer 222. This is best shown in FIGS. 10a through 10d. Alternatively, only the second portion may be provided with the cavities such that the additional anchorage is provided by adhesion to the cortical layer 220 alone.

In yet a further alternative, rather than delivering a bio-compatible material 801, the channel 206 may be arranged to house engaging projections which, when actuated, project out of the cavity or cavities 810 defined in at least one of the first and second portions 806, 808. For example, the threaded body 804 may be provided with lateral cavities or ports located between the threads such that when an internal actuator of the screw is rotated (or otherwise actuated) the projections rotate and project out the circumferential wall to engage the surrounding bone (e.g. like thorns).

In an alternative embodiment, the bio-compatible material 801 may be stored in the in the cavity until such times as it is required to be injected into the cancellous bone layer. For example, a plunger type configuration may be provided within the screw such that once the plunger is actuated a seal located at the distal end of the screw thread (which is provided to prevent the bio-compatible material from setting when not in use) may break to allow the bio-compatible material to be forced into the cancellous layer.

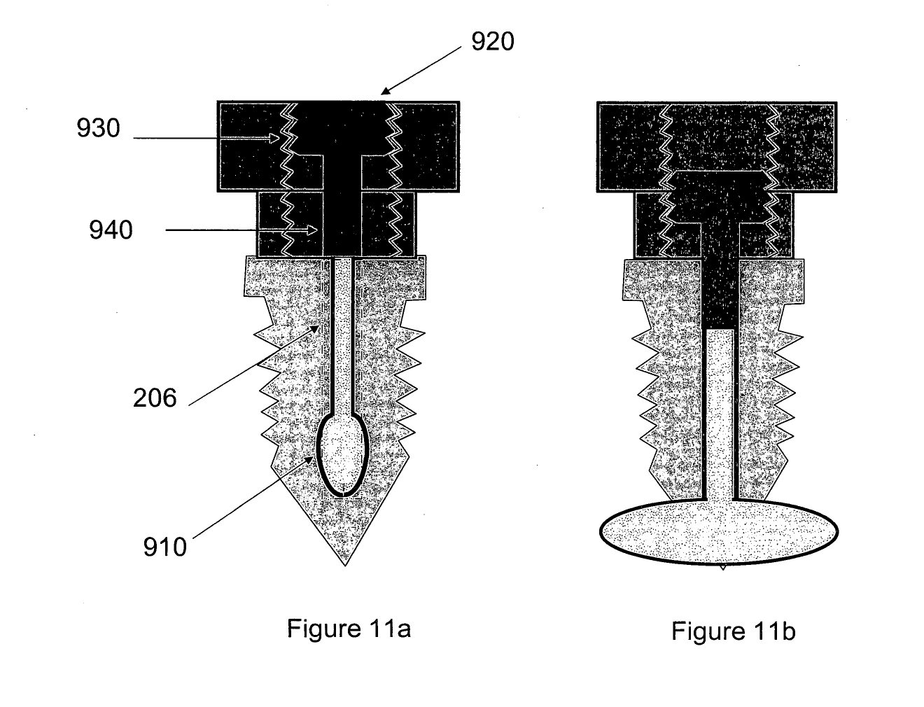

A still further retention mechanism is shown in FIG. 11. According to this embodiment the cavity 206 is used to retain a deformable bladder 910 filled with a bio-compatible fluid, such as saline. In use, at least part of the bladder 910 is arranged to extend into the cancellous bone layer 222, specifically to fill a space within the trabeculi net formed therein.

To facilitate insertion and withdrawal of the bladder 910, the mini-screw implements a plunger 920. The plunger 920 comprises a head 930 that is received by a threaded head portion of the mini-screw (i.e. in a similar configuration to that shown in FIGS. 6 & 7). A stem 940 coupled to the bladder 910 is arranged to extend into the cavity 206, such that rotation of the head 930 causes the bladder 910 to either extend into or out of the cancellous bone layer.

As mentioned above with reference to FIG. 2b, the mini-screw described herein can be coupled to an ancillary to facilitate tooth movement. The ancillary may, for example, comprise a magnetic system providing a plurality of magnetic attachments that work on one another to achieve forces (e.g. horizontal, rotational, vertical, a combination of forces, etc.) for suitably moving the tooth/teeth or correcting an orthodontic condition. According to FIG. 2b, a first magnetic attachment 106 of the magnetic system is coupled to the mini-screw 100 by way of a rigid arm 105. The first magnetic attachment 106 comprises a first magnet which is operable to attract an opposing magnet provided on a second magnetic attachment 108 bonded to the patient's canine tooth 101. Irrespective of the forces at play, the mini-screw 100 is able to be firmly held in place by virtue of the anchorage wires 302 that engage the cortical bone inner wall 220 to thereby prevent the screw from being loosened (e.g. unscrewed or pulled out).

Besides being used to minimise gaps between a patient's teeth or for correcting jaw alignment, mini-screws in accordance with embodiments described herein may be used for a range of other orthodontic applications. For example, the devices could be used to impart the desired correctional forces for intrusion, extrusion, rotation, torque and tip control orthodontic procedures, as will be understood by persons skilled in the art.

It will be understood that the various anchorage mechanisms described with reference to FIGS. 3 to 11 may also be suitable for use with conventional length mini-screws (i.e. having thread lengths greater than 6 mm).

Further, it will be understood by persons skilled in the art that embodiments may find application outside of the orthodontic field. For example, embodiments could be applied in other fields of dentistry or medicine such as orthopaedics, prosthodontics (for example in implantology) automotive, aeronautical, or mechanical fields whereby a screw is required to be anchored in a wall comprising an anchorage layer and a secondary non-anchorage layer disposed immediately behind the anchorage layer. One example finds application in the orthopaedic prosthetics filed whereby an artificial joint may be fixed to a long bone (e.g. femur) of a patient. Further the invention may be applied in the field of animal therapeutics and orthopedics.

In one embodiment, the invention can be applied in the field of dentofacial orthopaedics. Presently this field utilises anchorage in the form extra oral devices or utilising surgically placed skeletal plates and fixation screws. The increased mechanical stability and retention properties of embodiments will permit the use of higher force levels required in orthopaedic treatment. This application may apply to both the dentofacial complex and other bony structures of the body.

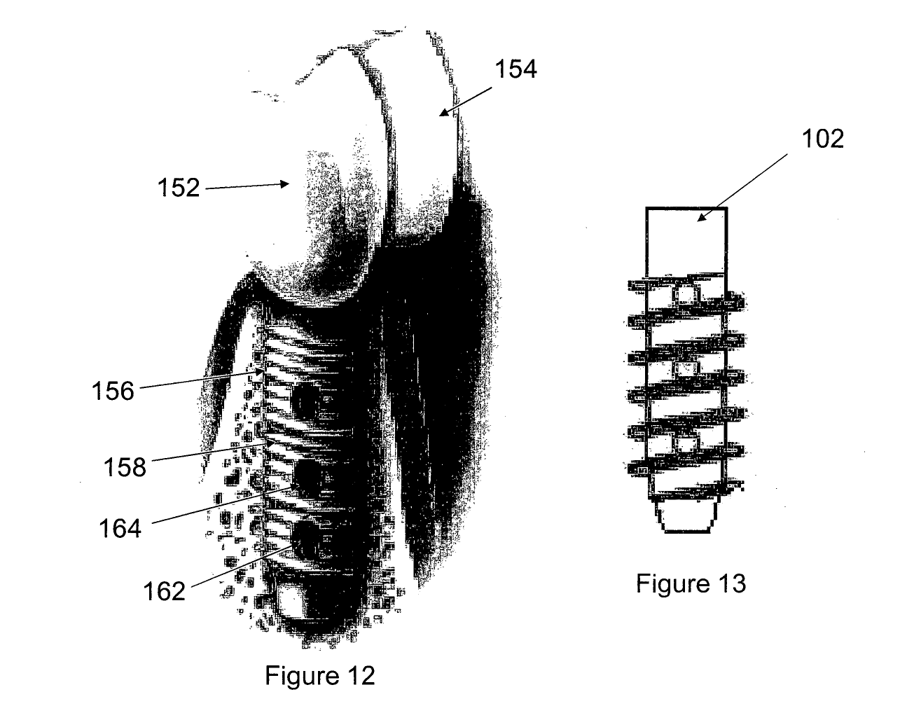

As mentioned above, embodiments also find application in the field of prosthodontics. For example, one embodiment may be used with a permanent dentoalveolar implants. The biocompatible material used in this application may be modified to be less rapidly resorbed than the orthodontic application. An example of such an embodiment is shown in FIGS. 12 and 13. As shown, the prosthodontic device is in the form of a screw 150 (although it will be understood that the implant may not be a screw and instead be in the form of a pin or other suitable implant) comprising a screw head 152 arranged to be coupled to a prosthodontic appliance 154 in the form of a tooth. The screw 150 further comprises a threaded body 156 for screwing into an anchorage bone layer 158. A channel 160 extends at least partially through the threaded body 156 and out of at least one void 162 located in a circumferential wall 164 of the threaded body. In use the channel 160 is arranged to deliver a substance (in this case resorbable cement, although other substances including treatment fluids etc could equally be delivered) to the anchorage layer. It will be understood that for pre-drilled embodiments, the substance could be introduced into the drill hole before the screw is screw into place therefore obviating the need to introduce the substance post location of the screw.

Further the invention may be used to increase the retention and stability of implant fixtures in patients with decreased bone density.

A non-biological application of the invention may be embodied in the form of a furniture screw that needs to be suitably anchored in a plasterboard wall for holding a picture frame. The type of anchorage mechanism utilised will depend on the application, but for the furniture screw example could take the form of the FIG. 8 embodiment where the non-biological settable material may be silicon, expandable foam, industrial/liquid concrete or other suitable setting agent.

A particular advantage arising through use of the aforementioned anchorage mechanisms is that in contrast to convention screw-anchorage devices, embodiments described herein are suitable for use where the adjacent rearward layer has some density (i.e. is not simply a hollow cavity).

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-57. (canceled)

58. An orthodontic mini-screw comprising:
a screw head arranged to be coupled to an ancillary member; and
a threaded body coupled to the screw head and having a first portion arranged to locate in a cortical bone layer of a patient's jaw bone and a second portion arranged to extend a predefined distance beyond the cortical bone layer and into an adjacent cancellous bone layer, such that a channel extends through the threaded body and out of at least one void located in the first portion, in use the channel being arranged to deliver a settable bio-compatible material into the cancellous bone layer to assist with anchorage of the mini-screw.

59. An orthodontic mini-screw in accordance with claim 58, wherein the at least one void is located in a circumferential wall of the threaded body.

60. An orthodontic mini-screw in accordance with claim 59, wherein the at least one void is located between a thread disposed on the threaded body.

61. An orthodontic mini-screw in accordance with claim 60, comprising a plurality of voids laterally displaced along the second portion of the threaded body.

62. An orthodontic mini-screw in accordance with claim 58, wherein a distal end of the threaded body comprises a solid cutting tip.

63. An orthodontic mini-screw in accordance with claim 58, wherein the second portion extends between 1 mm and 4 mm into the cancellous bone layer.

64. An orthodontic mini-screw in accordance with claim 63, wherein the second portion extends between 2 mm and 3 mm into the cancellous bone layer.

65. An orthodontic mini-screw in accordance with claim 58, wherein the threaded body has a total length of between 5 mm and 7 mm.

66. An orthodontic mini-screw in accordance with claim 58, wherein at least one additional void is provided on a circumferential wall of the first portion.

67. An orthodontic mini-screw in accordance with claim 58, wherein the at least one void is located at a bone engaging end of the threaded body.

68. An orthodontic mini-screw in accordance with claim 58, wherein the channel extends the length of the threaded body such that a needle of a syringe containing the bio-compatible material can be inserted into the cavity for injecting the bio-compatible material into the cancellous bone layer.

69. An orthodontic mini-screw in accordance with claim 58, wherein the channel has a retentive inner wall that allows bio-compatible material, once set, to be retained thereto.

70. An orthodontic mini-screw in accordance with claim 58, wherein the bio-compatible material comprises a resorbable material.

71. An orthodontic mini-screw comprising:
a screw head arranged to be coupled to an ancillary coupling member; and
a threaded body, the length of which substantially corresponds to a thickness of a first layer of cortical bone in which the mini-screw is to be located.

72. An orthodontic mini-screw in accordance with claim 71, wherein the length of the threaded body is equal to or less than 5 mm.

73. An orthodontic mini-screw in accordance with claim 72, wherein the length of the screw ranges from between 2 mm to 4 mm.

74. An orthodontic mini-screw in accordance with claim 71, wherein the threaded body is arranged to receive an anchorage device which, in use, engages a wall of the cortical bone to assist with anchorage of the mini-screw.

75. An orthodontic mini-screw in accordance with claim 71, wherein the anchorage device comprises at least one anchorage wire which is arranged to extend out of an opening in a distal end of the screw body and into a second non-cortical bone layer before engaging an internal cortical bone wall.

76. An orthodontic mini-screw in accordance with claim 75, wherein the opening extends into a peripheral wall of the threaded body to allow the anchorage wire(s) to immediately extend out of the threaded body into the second layer.

77. An orthodontic mini-screw in accordance with claim 75, wherein the second bone layer is composed of cancellous bone.

78. An orthodontic mini-screw in accordance with claim 76, wherein the second bone layer is composed of cancellous bone.

79. An orthodontic mini-screw in accordance with claim 75, wherein the at least one anchorage wire is formed of super-elastic shape memory material which allows the wire, having passed out of the distal end, to form a shape which is suitable for engaging the internal wall with minimal intrusion into the cancellous bone.

80. An orthodontic mini-screw in accordance with claim 79, wherein the elasticity of the super-elastic wire is selected so as to allow the wire to take the desired shape with minimal diversion during emplacement.

81. An orthodontic mini-screw in accordance with claim 80, wherein the shape is a generally parabolic shape.

82. An orthodontic mini-screw in accordance with claim 79, wherein the super-elastic shape memory material is NiTi.

83. An orthodontic mini-screw in accordance with claim 80, wherein the super-elastic shape memory material is NiTi.

84. An orthodontic mini-screw in accordance with claim 81, wherein the super-elastic shape memory material is NiTi.

85. An orthodontic mini-screw in accordance with claim 74, wherein the anchorage device comprises at least one projection which, in use, extends out of a circumferential wall of the threaded body to thereby engage the internal cortical bone wall.

86. An orthodontic mini-screw in accordance with claim 71, wherein the screw head includes a cavity arranged to retain a non-engaging end of the anchorage wire(s).

87. An orthodontic mini-screw in accordance with claim 86, wherein a head portion of the cavity is threaded and such that the non-engaging end of each anchorage wire is coupled to a retaining screw that is operable to be screwed in and out of the threaded cavity to thereby engage and disengage, respectively, the anchorage wires.

88. An orthodontic mini-screw in accordance with claim 87, further comprising a collar located between the threaded body and screw head, such that the threaded head portion is at least partly located within the collar.

89. An orthodontic mini-screw in accordance with claim 71, wherein the threaded body includes a cavity which is open at a bone engaging end, the cavity being arranged to receive and deliver a settable bio-compatible material into a cancellous bone layer behind the cortical bone, to thereby assist with anchorage of the mini-screw.

90. An orthodontic mini-screw in accordance with claim 89, wherein the cavity extends the length of the threaded body such that a needle of a syringe containing the bio-compatible material can be inserted into the cavity for injecting the bio-compatible material into the cancellous bone layer.

91. An orthodontic mini-screw in accordance with claim 89, wherein the cavity has a retentive inner wall that allows bio-compatible material, once set, to be retained thereto.

92. An orthodontic mini-screw in accordance with claim 90, wherein the cavity has a retentive inner wall that allows bio-compatible material, once set, to be retained thereto.

93. An orthodontic mini-screw in accordance with claim 71, wherein the threaded body includes a cavity which is open at a bone engaging end and arranged to receive a deformable bladder filled with a bio-compatible fluid, in use at least part of the bladder arranged to extend into a cancellous bone layer behind the cortical bone layer, to thereby assist with anchorage of the mini-screw.

94. An orthodontic mini-screw in accordance with claim 93, further comprising a plunger arranged to force the bladder into the cortical bone layer.

95. An orthodontic mini-screw in accordance with claim 94, wherein the plunger is coupled to the head of the mini-screw.

96. An orthodontic mini-screw in accordance with claim 95, wherein the plunger comprises a head that is received by a threaded head portion of the mini-screw and a stem coupled to the bladder and arranged to extend into the cavity, such that rotation of the head causes the bladder to either extend into or out of the cancellous bone layer.

97. An orthodontic treatment method requiring cortical bone anchorage comprises:
screwing a mini-screw as claimed in claim 58 into an insertion location using the screw head; and
passing the settable bio-compatible material through the channel and into the cancellous bone layer.

98. An orthodontic treatment method requiring cortical bone anchorage comprises providing a mini-screw comprising:
a screw head arranged to be coupled to an ancillary coupling member for affecting the treatment; and
a threaded body, the length of which substantially corresponds to a thickness of the cortical bone at an insertion location; and
screwing the mini-screw into the insertion location.

99. An orthodontic treatment method as claimed in claim 98, comprising the further step of actuating an anchorage device received by the threaded body such that it engages an internal wall of the cortical bone to assist with anchorage of the mini-screw.

100. An orthodontic treatment method as claimed in claim 99, wherein the anchorage device comprises at least one anchorage wire which is arranged to extend out of an opening in a distal end of the threaded body and into a second non-cortical bone layer before engaging the internal cortical bone wall.

101. An orthodontic treatment method as claimed in claim 100, wherein the opening extends into a wall of the threaded body to allow the anchorage wire(s) to immediately extend out of the threaded body into the second layer.

102. An orthodontic treatment method as claimed in claim 100, wherein the second non-cortical bone layer is composed of cancellous bone.

103. An orthodontic treatment method as claimed in claim 100, wherein the at least one anchorage wire is formed of super-elastic shape memory material which allows the wire, having passed out of the distal end, to form a shape which is suitable for engaging the internal wall with minimal intrusion into the cancellous bone layer.

104. An orthodontic treatment method as claimed in claim 103, wherein the shape is a generally parabolic shape.

105. An orthodontic treatment method as claimed in claim 103, wherein where two wires are utilised, the respective distal ends divert so as to substantially form an oval shape within the second layer.

106. A screw comprising:
a screw head;
a threaded body for screwing into an anchorage layer; and
an anchorage device comprising at least one anchorage wire arranged, in use, to extend out of an opening in the threaded body and into a space behind the anchorage layer, such that at least part of the anchorage wire engages an internal wall of the anchorage layer to thereby assist with anchorage of the screw.

107. A screw comprising:
a screw head; and
a threaded body for screwing into an anchorage layer, wherein the threaded body includes a cavity extending therethrough, in use, arranged to receive and deliver a settable substance into a space behind the anchorage layer, to thereby assist anchorage of the screw.

108. A screw in accordance with claim 107, wherein the substance comprises at least one of silicon, expandable foam and industrial cement.

109. A screw in accordance with claim 107, wherein the cavity extends the length of the threaded body such that a needle of a syringe containing the settable substance can be inserted into the cavity for injecting the substance into the space.

110. A screw in accordance with claim 107, wherein the cavity has a retentive inner wall that allows the substance, once set, to be retained thereto.

111. A screw comprising:
a screw head; and
a threaded body for screwing into an anchorage layer, wherein the threaded body includes a cavity arranged to receive a deformable bladder arranged to be filled with a fluid, in use, at least part of the bladder arranged to extend into a space behind the anchorage layer, to thereby assist with anchorage of the screw.

112. A screw in accordance with claim 111, further comprising a plunger arranged to force the bladder into the space.

113. A screw in accordance with claim 112, wherein the plunger is coupled to the head of the mini-screw.

114. A screw in accordance with claim 113, wherein the plunger comprises a head that is received by a threaded head portion of the mini-screw and a stem coupled to the bladder and arranged to extend into the cavity, such that rotation of the head causes the bladder to either extend into or out of the space.

115. A mini-screw arranged to be located in a layer of cortical bone, the mini-screw comprising:
a screw head arranged to be coupled to an ancillary coupling member; and
a threaded body providing an anchorage device which, in use, engages an internal wall of the cortical bone to assist with anchorage of the mini-screw.

116. A mini-screw as claimed in claim 115, wherein the anchorage device comprises at least one anchorage wire which is arranged to extend out of an opening in a distal end of the threaded body and into a second non-cortical bone layer, before engaging the first layer's internal wall.

117. A mini-screw in accordance with claim 58, wherein the bio-compatible material/fluid comprises at least one of bone graft material, calcium phosphate, tri calcium phosphate, hydroxyapatite cement and bone morphogenetic protein BMP 2 or 4 containing grafting materials.

118. A prosthodontic implant comprising:
a head arranged to be coupled to a prosthodontic appliance; and
a body for locating into an anchorage layer, wherein the body includes a channel extending at least partially therethrough and out of at least one void located in a circumferential wall of the body, in use the channel arranged to deliver a substance to the anchorage layer.

119. An implant in accordance with claim 118, wherein a thread is located on the circumferential wall of the body.

120. A screw in accordance with claim 118, wherein the substance is a bio-compatible material that assists in anchorage of the implant.
